(19) United States
(12) Patent Application Publication
(10) Pub. No.: US 2024/0156382 A1
(43) Pub. Date: May 16, 2024

(54) DIAGNOSIS AND MONITORING MEDICAL TREATMENT EFFECTIVNESS FOR ANXIETY & DEPRESSION DISORDERS

(71) Applicant: Iluria Ltd., Hod Hasharon (IL)

(72) Inventors: Birkat Klimshtein Levy, Hod Hasharon (IL); Ran Izraeli, Tel Aviv (IL); Hagay Levy, Hod Hasharon (IL)

(73) Assignee: Iluria Ltd., Hod Hasharon (IL)

(21) Appl. No.: 18/551,002

(22) PCT Filed: Mar. 20, 2022
(86) PCT No.: PCT/IB2022/052516

§ 371 Date: Sep. 18, 2023

Related U.S. Application Data
(60) Provisional application No. 63/164,673, filed on Mar. 23, 2021.

(51) Int. Cl. A61B 5/16; A61B 5/00; G16H 50/20

(52) U.S. Cl. CPC A61B 5/165; A61B 5/4848; G16H 50/20

(57) ABSTRACT

A method and a system are provided for taking physiological markers measurements of patients who have anxiety or depression. Mathematical analysis (e.g., pattern recognition, machine learning and AI algorithms) of the physiological markers measurements is used to create a unique personal prediction model and data set for an individual patient. The unique personal data set is used to diagnose and monitor a particular problem of the individual patient associated with anxiety or depression, prevent potential overdosing or to recommend a treatment for a particular problem of the individual patient associated with anxiety or depression, or to predict an outcome of a treatment for a particular problem of the individual patient associated with anxiety or depression.

### FIELD OF THE INVENTION

The present invention generally relates to a method of analyzing a subject for Anxiety and Depression Disorders (Anxiety & Depression), and particularly, but not limited to, determining Anxiety & Depression diagnosis and the effectiveness of the medication, appropriate dosage and specifically combinations of various medications, taken to counteract Anxiety & Depression or other related clinical treatments.

### BACKGROUND OF THE INVENTION

Anxiety contains are a group of mental disorders, in which patients experience intense feelings of Anxiety that appear at an inappropriate time, do not pass in a short time and interfere with the management of a normal life. Anxiety disorder can be classified as generalized anxieties, which do not depend on a specific stimulus, or a stimulus that will not cause Anxiety in most people; And situational anxieties resulting from a condition that seems to most humans to be threatening, although not everyone feels anxious as a result of these conditions. An example of situational Anxiety is “exam anxiety” which appears in different intensities in different people.

Depression is a mental disorder, characterized by a widespread and persistent pattern (usually weeks and months) of poor mood, accompanied by low self-esteem and loss of interest and enjoyment of enjoyable activities, anxiety, sleep and appetite disorders, lack of energy, pessimistic thoughts to varying degrees, for life and suicide, decreased concentration and memory, and significant impairment of function. Sometimes it also appears in a feeling of emptiness and lack of emotion. This collection of symptoms was given its professional name, described and classified as a mood disorder in the 1980 edition of the American Psychiatric Association (DSM) Diagnostic Manual.

The treatment for Anxiety and Depression is similar, containing both similar medical interventions and a range of psychological treatments. As for intervention which involves medications, which is the focus of this proposed invention, both Anxiety & Depression current treatment has three basic phases:
a. Initial diagnosis and setting up initial recommended treatment (e.g., type of medication and dosage);
b. Overdosing analysis and potentially adding supplementary medical treatment to prevent unwanted results (e.g., serotonin overdosing which may lead to potential psychosis). This potential risk can be mitigated by creating a combination of medications using additional anti psychosis medications to prevent hyper/psychotic outburst; and
c. Ongoing treatment monitoring is needed, as the drug efficacy changes over time and side effects may occur (e.g., serotonin overdosing and psychosis as mentioned above); for these reasons, the treatment should be adjusted from time to time.

Anxiety and Depression diagnosis and monitoring is done by physicians using various methods, mostly subjective. Generally, it involves interviewing the patients, parents, teachers, and school staff, etc. The specialist will then incorporate and analyze all of the data obtained and make a decision based on his/her findings. In some cases, the diagnosis and monitoring processes involve ratings or scores for Anxiety & Depression level.

Anxiety & Depression are difficult disorders to diagnose. Moreover, once a patient is diagnosed, managing the ongoing process using combination of different medications is completely trial-and-error based, relying on physician's previous experience and post factum event analysis.

In addition to the above, both diagnosis and monitoring phases do not relate to different patient clusters. Currently, other than age and body weight classes, Anxiety & Depression patients are not grouped into clusters with similar background and physiological attributes within each cluster. As a result, the initial drug allocation and ongoing adjustments are based solely on the patient's age and weight, with no other additional relevant attributes, with sporadic preliminary calibrations based on the physician's previous experience.

There is thus a need for a passive, objective, inexpensive, and reliable technique for determining Anxiety & Depression diagnosis and continuous monitoring and the effectiveness of the medication, appropriate dosage taken, detect overdosing to monitor and counteract Anxiety & Depression and other clinical treatments.

### SUMMARY OF THE INVENTION

The present invention seeks to provide a method, and a tracking and evaluation algorithmic system, analyzing changes in various physiological markers, for determining continuous Anxiety & Depression diagnosis, monitoring, predicting the need for a combination of different medications and preventing under/over dosing. The method and system include: A) allocating the patient to one of pre-determined groups of Anxiety & Depression patient clusters; B) personalized analysis of their medical treatment effectiveness to prevent symptoms of Anxiety & Depression; and C) prevent overdosing and analyzing the need for combination of more than one medication (i.e. to prevent possible hyper/psychotic outburst due to serotonin overflow). Unlike the current trial and error mitigation processes, using subjective inputs and self-reporting, using our novel approach these three processes will be done using objective mathematical analysis of changes in a series of physiological markers. The mathematical analysis will be done using trained machine learning based search patters designated for Anxiety & Depression analysis and then personally calibrated per group of patients.

As opposed to the prior art, the physiological markers measurements are used to define the probability of the treatment success in lowering the Anxiety or Depression level and the probability for the need of additional medications in parallel, to prevent potential overdosing, all of these by analyzing an ensemble of physiological markers. The process will include: 1) defining pre-determined personal groups of patients; and B) create a personal profile, baseline and prediction pattern per patient to analyze both under dosing and overdosing situations. The patients in each cluster have similar attributes (e.g., gender, age, comorbidities) and physiological markers measurements that are similar, so that within each cluster biomarker variably will be significantly smaller than the between clusters variability.

The innovative process will include:
Allocation to patient cluster: providing patient cluster based analysis of each specific treatment success with respect to under/over dosing;
Ongoing treatment effectiveness analysis: by calibrating the generic model assigned to each cluster to create unique treatment effectiveness personal model. This will be done by using machine and deep learning techniques; and
Overdosing monitoring: by creating another set of machine learning based personal models, analyzing changes in physiological markers, to predict potential overdosing and the need for either a sole type of medication or a combination of medications.

### BRIEF DESCRIPTION OF THE DRAWINGS

The present invention will be understood and appreciated more fully from the following detailed description, taken in conjunction with the drawings in which:

FIG. 1 demonstrated the data flow concept: retrieving physiological data (using a smart wearable device), analyzing the data with our machine learning based engine and assigning various analysis reports to the different stakeholders.

FIG. 2 demonstrates quantified continuous monitoring, with and without medical treatment.

FIG. 3 demonstrates aggregated quantified data with regards to optional dosing (in order to avoid crossing the hyper/psychotic threshold), using data clustering generated from an ensemble of machine learning based search patterns:

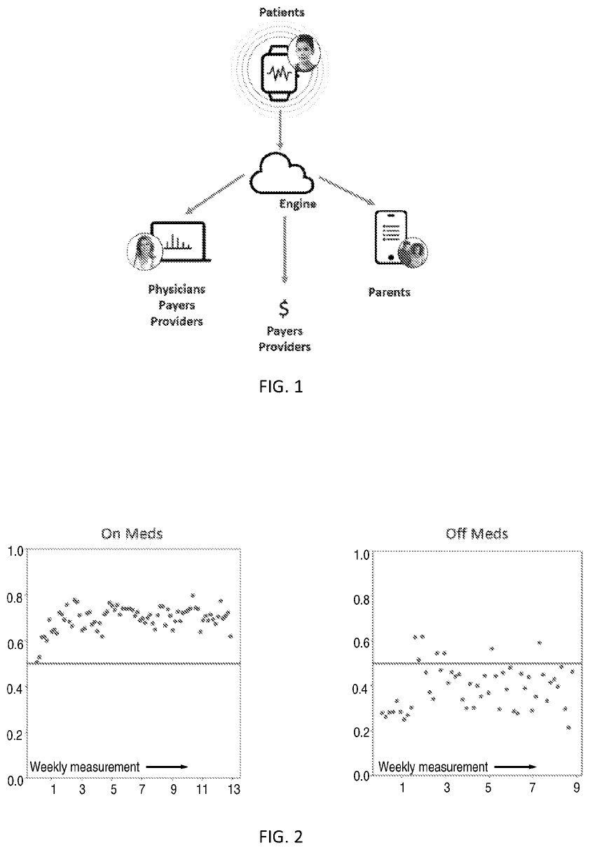

### DETAILED DESCRIPTION OF EMBODIMENTS

As mentioned above, in contrast to the prior art, the present invention uses mathematical analysis of biomarker measurements to define ongoing pattern efficacy, clusters of patients and, for each patient, to create a personal profile, baseline and prediction patterns for both under and overdosing. The unique personal models are calibrated by machine and deep learning techniques which are specifically assigned per patient, for the provision of output that assess Anxiety & Depression.

The personal method may include the followings steps:
A: Cluster allocation:
   Using patients' measured biomarker data to build an ensemble of predictive models for clinical Anxiety & Depression diagnosis tools used by physicians. This step uses biomarker data measured during the clinical diagnosis process, and would use a host of pattern recognition, machine learning and AI algorithms.
   Using pattern recognition, machine learning and AI algorithms to cluster patients based on external features (such as gender, age, and comorbidities) and internal features (biomarker data), and to modify the predictive model to each cluster.
B: Personal baseline calibration and continues monitoring of medical treatment effectiveness:
   Using pattern recognition, machine learning and AI algorithms to calibrate model features (e.g., physiological markers weighting and/or selecting the importance of a particular physiological feature) and tailor a predictive model to each individual, based on its cluster association and on its unique combination of external and internal features.
   Using the trained personalized predictive model to automatically and passively monitor patients for their response to Anxiety & Depression medical treatment.
C: Overdosing prevention:
   Using another layer of designated models the system will monitor the patient to prevent overdosing and potential mania bursts.
D: Ongoing calibration:
   The predictive models are automatically updated and improved (continuous learning) with more biomarker data measured for each patient.

The method also assesses the objective impact of other treatment options for Anxiety & Depression patients, such as CBT, nutrition and various behavioral treatments.

The output of the method is achieved by demonstrating personal machine learning based prediction models and personal baseline of the individual with and without effective treatment. This output supports physicians' evaluation of treatment success and provides additional, ongoing analysis which recommends possible advantageous treatment modifications.

The method uses pattern recognition, machine learning, AI algorithms, and other techniques. It is based on physiological marker measurements (described below) and external information (such as gender, age, etc.). A predictive model is designed upon collection of all samples, then modified within each cluster of patients, and then further modified to match the specific personal pattern of every patient, with separated models for treatment analysis and overdosing prediction. Eventually, every individual patient is characterized by a unique, personalized, predictive model.

The following is a description of one non-limiting embodiment of the method and system of the invention.

First, a combination of the following of biomarkers' measurements may be automatically collected and recorded by a device, such as a wearable device:

| | |
|---|---|
| PPG | nano watt |
| Inter beat interval | Sec |
| Heart rate (BPM) | Sec |
| Skin temperature | Celsius ° C. |
| Accelerometer | gravity (g) |
| (or motion sensor) | |
| Heart Rate Variability | hertz (Hz) |
| Gyro | degrees per second (°/s) |
| Respiratory rate | BRA |
| Blood pressure | mmHg |
| EEG | μV |
| Skin conductance | Hz |
| ECG | mV/mS |
| Blood oxygen saturation | SaO₂ |
| Systemic vascular resistance | PRU |

Afterwards, patients may be assigned to a pre-defined cluster using the physiological markers and attributes. For example, initial ongoing physiological markers measurement, together with patient's attributes (e.g., girl, 15 years old, Afro-American, with birth difficulties) may be used to assign a patient into a designated cluster. The method may start with a certain number of clusters, and in parallel there may be ongoing cluster calibration and addition of new clusters.

The methods for clustering and for building predictive models may be based on an ensemble of models, including linear models (e.g., Fisher Discriminant Analysis and Linear Discriminant analysis) and nonlinear ones (e.g., neural network classifiers, and random forests). The ensemble of models may be adjusted and constantly re-trained whenever more patient data become available.

The method may also include measuring physiological markers in parallel to physician diagnostics. For example, the measurement may be carried out together with the clinical evaluation to associate physiological markers with Anxiety & Depression scoring.

The method may also include calculation of a personal calibration model. The method may assign a personal calculation model to setup baseline and ongoing prediction models.

The method may also include setting up personalized physiological markers weightings and significance level. For example, these may include personal physiological markers weighting and/or selecting the importance of a particular feature while assessing non-linear models.

The method may also include both monitoring ongoing medical treatment and/or preliminary Anxiety & Depression diagnosis. This may include utilizing personal prediction patterns and performing ongoing analysis as well as overdosing prediction, using machine learning based process.

The method may also include ongoing cluster calibration and shifts between clusters. For example, there may be ongoing evaluation of the patient to decide whether he/she should shift between clusters (e.g., age group changed from 7-10 to 11-14), or there may be ongoing creation of new clusters or sub-clusters.

Personal Process—the Significance of Personal Modeling:

The significance of personal modeling is embedded in the unique physiological attributes of each patient. Mathematically analyzing changes in physiological markers will include personal modeling and model calibration per each patient. Such process will include assigning a most suitable machine learning based search pattern that will best distinguish between on/off treatment cases (i.e., the gap between gap on/off treatment cases and the overall treatment significance and effectiveness). Such personal methods may include ready-made search patterns, ensemble of more than one patterns or a designated machine learning based model.

Technical Process:

All of the physiological markers measurements are automatically collected and recorded by a wearable device. Data may be transformed to a centralized hub. Data may be stored within the wearable device memory for later downloading. Thereafter, the data may be transmitted or uploaded to a digital data repository (cloud or other) and analyzed by implementing machine and deep learning techniques.

System output is a translation of the processed analysis into medical indicators and status reports and is provided to physicians through a medical web application, and to the individuals (patient, child, parents, etc.) through a mobile application.

Analysis is seamlessly transferred to the individual's smartphone (or to other communication device), or via a transmitting, add-on dedicated unit for real-time data transmission.

The web application provides the physicians the results of the analysis which include continuous diagnosis and monitoring of Anxiety & Depression, which may include indication of effective treatment, dosing effectiveness, actual duration of effect, long term patterns and trends and other clinical aspects (e.g., effectiveness of other clinical treatments, and effect on the quality of sleep), predictions related to recommended treatment changes, level of patient medication usage, adherence and overall treatment effectiveness snapshots. Thus, the method provides medical professionals big data insights into real-life patient clinical and behavioral patterns.

In addition, the method may use patient clustering analysis to provide deep learning-based recommendations (e.g., preliminary type of recommended medication to be allocated to each cluster for the initial treatment process).

The application provides a clear view of the medication term or effectiveness during both the daily and intra-day periods, and helps prepare and better manage the activities of the individual, using predictions of Anxiety & Depression symptoms' levels.

System's Elements/Components:

The system implementing the method is an IoT platform which may include a user-friendly wearable device. The wearable device may include, without limitation, a sensor hub that include sensors for all biomarkers mentioned in the above table, a user interface (e.g., including graphics, sound and vibration methods of interface), and a software application. For example, without limitation, the application may operate the related services and processes to read the sensors, perform the analysis and send the real time feedback through the user interface to the user. This software application may also communicate with the cloud-based software on a real time or a periodic basis, to update the data measured from the sensors in the device.

The system may further include a cloud-based SW tool or other data repository, which aggregates and analyzes the sensors data, updates related algorithms and generates output in dash boards and reports to stakeholders.

The system may further include a web application interface for medical professionals—providing output on diagnosis of Anxiety & Depression, indication of medication and dosing effectiveness, predictions of recommended treatment changes, adherence and overall treatment effectiveness snapshots.

The system may further include a mobile application interface for users (individuals who have Anxiety & Depression and stakeholders (e.g., parents) providing a real time view on the individual's Anxiety & Depression condition throughout the day, including medication cycle impact and phasing out timing, and predictions of Anxiety & Depression personal patterns.

Method Outputs and Example of the Importance of Personal Analysis:

Tests of the methods of the invention have demonstrated their efficacy, resulting in outputs significantly correlated to the demonstrated clinical results, as indicated by physicians.

The following section provides examples of personal, ongoing analysis using biomarkers.

### EXAMPLES: OUTPUT SAMPLES

### Example 1

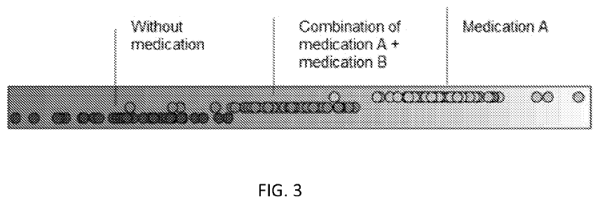

overdosing mitigation (shown in FIG. 3): an example case of a child treated with Prizma (Fluoxetine), later switched to a combination of Prizma+Ariply-classification outputs clearly demonstrate the overdosing analysis of two medication mixture, aligned with physician's clinical observations (Prizma: overdosing, Prizma+Ariply: effective treatment). The classification is based on multiple physiological markers measurements combined and a personalized formula determining the significance of each marker for this specific patient.

What is claimed is:

1. A method for dealing with anxiety and depression disorders comprising:
taking physiological markers measurements of patients who each have anxiety or depression;
using said physiological markers measurements to create a cluster of anxiety or depression patients, wherein the patients in said cluster have similar attributes and physiological markers measurements, so that within each cluster physiological markers variably will be significantly smaller than the between clusters variability; and
processing differences between said physiological marker measurements of the patients to create a unique personal data set for an individual patient; and
using the unique personal prediction models and data set to A) diagnose and monitor a particular problem of said individual patient associated with anxiety or depression and B) predict treatment efficacy, potential overdosing, or to recommend a treatment for a particular problem of said individual patient associated with anxiety or depression, or to predict an outcome of a treatment for a particular problem of said individual patient associated with anxiety or depression.

2. The method according to claim 1, wherein the step of processing differences is done by pattern recognition, machine learning or AI algorithms.

3. The method according to claim 1, comprising steps as follows:
assigning other patients to one of various patients' cluster depending on physiological markers measurements of said other patients; and
using pattern recognition of differences between the physiological markers measurements of said other patients to create a unique personal data set for an individual patient of said other patients; and
using additional personal pattern recognition models of differences between the physiological markers measurements to analyze potential over dosing

4. The method according to claim 3, wherein the unique personal data set further comprises calibration and calculation of a personal baseline.

5. The method according to claim 1, further comprising performing ongoing treatment, including delivering personal analysis of medical treatment effect and predictions using a personal pattern based on the unique personal data set.

6. The method according to claim 1, further comprising performing ongoing cluster calibration using automation machine learning and shifts between clusters.
